(19) United States
(12) Patent Application Publication
(10) Pub. No.: US 2026/0240971 A1
(43) Pub. Date: Aug. 20, 2026

(54) COMPOSITIONS FOR TREATMENT OF DIFFUSE INTRINSIC PONTINE GLIOMA

(71) Applicant: UNIVERSITY OF FLORIDA RESEARCH FOUNDATION, INCORPORATED, Gainesville, FL (US)

(72) Inventors: Elias Sayour, Newberry, FL (US); James Andrew McGuiness, Gainesville, FL (US); Hector Ruben Mendez-Gomez, Gainesville, FL (US); Duane A. Mitchell, Gainesville, FL (US)

(21) Appl. No.: 19/434,197

(22) Filed: Dec. 29, 2025

Related U.S. Application Data
(63) Continuation of application No. 17/607,495, filed on May 1, 2020, now Pat. No. 12,558,407.
(60) Provisional application No. 62/842,525, filed on May 2, 2019.

(51) Int. Cl. A61K 39/00; A61P 35/00; C07K 14/705

(52) U.S. Cl. CPC A61K 39/001114; A61K 39/0011; A61P 35/00; C07K 14/70539; C07K 14/70596; A61K 2039/53; A61K 2039/55555; A61K 2039/80; C07K 2319/00

(57) ABSTRACT

Provided herein are compositions comprising a liposome comprising RNA molecules and a cationic lipid, wherein the RNA molecules encode at least one MHC Class II epitope of a mutant Histone 3 (H3) protein comprising a K27M mutation and optionally at least one MHC Class I epitope of the mutant H3 protein. In exemplary embodiments, the RNA molecules comprise a sequence of SEQ ID NO: 12 or 14. Methods of increasing central memory T cells, increasing an immune response, or treating a diffuse midline glioma (DMG), in a subject are provided herein. In exemplary embodiments, the methods comprise administering to the subject the compositions provided herein.

### CROSS-REFERENCE TO RELATED APPLICATIONS

This application is a continuation application based on U.S. patent application Ser. No. 17/607,495, filed Oct. 29, 2021, which is a U.S. national phase under 37 C.F.R. 371 of International Patent Application No. PCT/US20/31096, filed May 1, 2020, which claims priority to U.S. Provisional Patent Application No. 62/842,525, filed May 2, 2019, the entire contents of each which are incorporated herein by reference.

### GRANT FUNDING DISCLOSURE

This invention was made with government support under grant number W81XWH-17-1-0510 awarded by the United States Army Medical Research Acquisition (ARMY/MRMC). The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 64,410 bytes file named 54160A_SeqListing.xml″; created on Dec. 22, 2025.

### BACKGROUND

Diffuse Intrinsic Pontine Glioma (DIPG) is an infiltrative and diffuse tumor found in the brain of pediatric patients. The median overall survival is 10-11 months and the 2-year survival rate is less than 10%. DIPG represents 10% to 20% of all pediatric brain cancer cases and is the number one cause of brain tumor-related deaths in children. Even when a diagnosed child survives past one year, they suffer from major neurological defects, as the growing tumor impinges on critical white matter tracts and local cranial nerve foci, leading to the onset of severe symptoms including ataxia, cranial nerve palsies, and long tract signs.

Treatment modalities for DIPG are limited. Because of its location and diffuse nature, DIPG cannot be addressed by surgical resection. Chemotherapies providing a therapeutic effect are few and far between. From 1984 to 2014, over 65 clinical trials using adjuvant chemotherapeutic agents have failed to demonstrate a therapeutic effect in patients with DIPG. While corticosteroids offer some relief by way of reducing peritumoral edema, this treatment does not improve the outcome of the DIPG patients. Not surprisingly, the 5-year survival rate of DIPG-diagnosed patients have remained essentially the same since 1962.

In view of the foregoing, there is a need for improved methods of treating DIPG.

### SUMMARY

Presented herein for the first time are data which shows an increase in central memory T cells having antigen-specificity for a mutant Histone H3 protein comprising a K27M mutation upon administration of a liposome comprising RNA molecules encoding an MHC Class II epitope (and optionally at least one MHC Class I epitope) of the mutant Histone H3 protein comprising the K27M mutation. Accordingly, the present disclosure provides compositions comprising a liposome comprising ribonucleic acid (RNA) molecules and a cationic lipid, wherein the RNA molecules encode at least one MHC Class II epitope (and optionally at least one MHC Class I epitope) of a mutant Histone 3 (H3) protein comprising a K27M mutation. In exemplary embodiments, the MHC Class II epitope comprises at least 5-10 consecutive amino acids of the sequence of MAPRKQLATKAARMSAPSTGGVKKPH (SEQ ID NO: 13) or RKQLATKAARMSAPSTGGVKK (SEQ ID NO: 15). In exemplary aspects, the MHC Class II epitope comprises at least 11-17 or at least 18-21 consecutive amino acids of the sequence of SEQ ID NO: 13 or SEQ ID NO: 15. In various instances, the MHC Class II epitope comprises at least 22-25 consecutive amino acids of the sequence of SEQ ID NO: 13 or SEQ ID NO: 15. In exemplary aspects, the MHC Class II epitope comprises the amino acid sequence of SEQ ID NO: 13 or SEQ ID NO: 15. In various aspects, the MHC Class II epitope comprises the sequence of ARM, RMS, or MSA. Optionally, the MHC Class II epitope comprises the sequence of MSAPS (SEQ ID NO: 16), RMSAP (SEQ ID NO: 17), ARMSA (SEQ ID NO: 18), AARMS (SEQ ID NO: 19), or KAARM (SEQ ID NO: 20). In exemplary aspects, the RNA molecules comprise at least a portion of the nucleotide sequence of SEQ ID NO: 12 which encodes the MHC Class II epitope. In some aspects, the RNA molecules comprise the nucleotide sequence of SEQ ID NO: 14 or SEQ ID NO: 21. Optionally, the RNA molecules comprise the nucleotide sequence of SEQ ID NO: 12.

In some aspects, the liposome comprises the cationic lipid, DOTAP. In some aspects, the liposome (a) has a zeta potential of about 30 mV to about 60 mV, optionally, about 40 mV to about 50 mV, (b) is about 50 nm to about 250 nm in diameter, optionally, about 70 nm to about 200 nm in diameter, (c) or a combination thereof. In various aspects, the composition comprises a plurality of liposomes, each liposome of which is about 50 nm to about 250 nm in diameter, optionally, about 70 nm to about 200 nm in diameter. The RNA molecules in some instances are complexed with the cationic lipid via electrostatic interactions. Optionally, the liposomes are prepared by mixing the RNA molecules and the cationic lipid at a RNA: cationic lipid ratio of about 1 to about 10 to about 1 to about 20, optionally, about 1 to about 15. In various aspects, the composition comprises about 10¹⁰ liposomes per mL to about 10¹⁵ liposomes per mL, optionally about 10¹² liposomes+10% per mL. In exemplary instances, the RNA molecules are mRNA and in exemplary aspects, each RNA molecule comprises a 5′-cap. In certain instances, the composition further comprises lysosome-associated membrane proteins (LAMPs). In exemplary aspects, the RNA molecules further encode a lysosome-associated membrane protein (LAMP). In various aspects, the RNA molecules encode a chimeric protein comprising a LAMP protein and the MHC Class II epitope of the mutant H3 protein and optionally at least one MHC Class I epitope.

The present disclosure also provides methods of generating a liposome comprising ribonucleic acid (RNA) molecules and a cationic lipid, wherein the RNA molecules encode at least one MHC Class II epitope (and optionally at least one MHC Class I epitope) of a mutant Histone 3 (H3) protein comprising a K27M mutation. In exemplary embodiments, the method comprises (i) in vitro transcribing a nucleic acid comprising a nucleotide sequence encoding the RNA molecules, (ii) chemically adding a 5′-cap to the in vitro transcribed RNA molecules, and (iii) mixing the RNA molecules comprising the 5′-cap with a cationic lipid. In exemplary aspects, the cationic lipid is DOTAP and/or the RNA molecules and cationic lipid are mixed at a RNA: lipid ratio of about 1 to about 10 to about 1 to about 20 (e.g., about 1 to about 15). In various aspects, the nucleotide sequence encoding the RNA molecules comprises the sequence of SEQ ID NO: 11 and optionally the nucleotide sequence encoding the RNA molecules is operably linked to a promoter, optionally a T7 promoter. In various aspects, the nucleotide sequence encoding the RNA molecules is flanked by a 5′ untranslated region (5′UTR) and a 3′ untranslated region (3′UTR). In some aspects, the nucleic acid comprises a sequence encoding a polyA tail. Optionally, the nucleic acid is a linearized plasmid (e.g., linearized DNA). Further provided are liposomes generated by the presently disclosed method of generating a liposome. In various instances, the liposome is formulated for intravenous injection.

RNA molecules encoding an MHC Class II epitope (and optionally at least one MHC Class I epitope) of a mutant Histone 3 (H3) protein comprising a K27M mutation are further provided herein. In exemplary embodiments, the MHC Class II epitope comprises at least 5-10 consecutive amino acids of the sequence of MAPRKQLATKAARMSAPSTGGVKKPH (SEQ ID NO: 13) or RKQLATKAARMSAPSTGGVKK (SEQ ID NO: 15). In various aspects, the MHC Class II epitope comprises at least 11-17 consecutive amino acids of the sequence of SEQ ID NO: 13 or SEQ ID NO: 15. In various instances, the MHC Class II epitope comprises at least 18-21 consecutive amino acids of the sequence of SEQ ID NO: 13 or SEQ ID NO: 15. In certain aspects, the MHC Class II epitope comprises at least 11-25 consecutive amino acids of the sequence of SEQ ID NO: 13 or SEQ ID NO: 15. In exemplary aspects, the MHC Class II epitope comprises at least 11-17 consecutive amino acids of the sequence of SEQ ID NO: 13 or SEQ ID NO: 15. In exemplary instances, the MHC Class II epitope comprises the sequence of SEQ ID NO: 13 or SEQ ID NO: 15. In various aspects, the MHC Class II epitope comprises the sequence of ARM, RMS, or MSA, optionally, wherein the MHC Class II epitope comprises the sequence of MSAPS (SEQ ID NO: 16), RMSAP (SEQ ID NO: 17), ARMSA (SEQ ID NO: 18), AARMS (SEQ ID NO: 19), or KAARM (SEQ ID NO: 20). In certain instances, the RNA molecule comprises at least a portion of the nucleotide sequence of SEQ ID NO: 12 which encodes the MHC Class II epitope, optionally, the RNA molecule comprises the nucleotide sequence of SEQ ID NO: 14 or SEQ ID NO: 21 or SEQ ID NO: 12. In exemplary instances, the RNA molecule further comprises a 5′ cap and/or a polyA tail and/or a nucleotide sequence encoding a lysosome-associated membrane protein (LAMP), optionally, wherein the RNA molecule encodes a chimeric protein comprising a LAMP protein and the MHC Class II epitope of the mutant H3 protein. Nucleic acids comprising a nucleotide sequence encoding the RNA molecule of the present disclosure are further provided herein. Compositions, e.g., pharmaceutical compositions, comprising the presently disclosed liposome, RNA molecule or nucleic acid, or a combination thereof, are additionally provided herein.

Methods of increasing in a subject the number of central memory T cells having antigen specificity for an epitope of a mutant Histone 3 (H3) protein comprising a K27M mutation are provided by the present disclosure. In exemplary embodiments, the method comprises administering to the subject a composition comprising the presently disclosed liposome, RNA molecule or nucleic acid, or a combination thereof, in an amount effective to increase the central memory T cells in the subject. Also, methods of enhancing in a subject an immune response against a diffuse midline glioma (DMG) expressing a mutant Histone 3 (H3) protein comprising a K27M mutation are provided. In exemplary embodiments, the method comprises administering to the subject a composition comprising the presently disclosed liposome, RNA molecule or nucleic acid, or a combination thereof, in an amount effective to increase the an immune response against the DMG in the subject. In exemplary aspects, the subject has a diffuse midline glioma (DMG) expressing a mutant Histone 3 (H3) protein comprising a K27M mutation or a predisposition to having the DMG. In various aspects, the subject has been treated for a DMG is in remission for the DMG. Methods of treating a subject with a diffuse midline glioma (DMG) expressing a mutant Histone 3 (H3) protein comprising a K27M mutation are also provided herein. In exemplary embodiments, the method comprises administering to the subject a presently disclosed composition in an amount effective to treat the DMG in the subject. In various aspects, the composition is systemically administered to the subject. In various aspects, the composition is intravenously administered to the subject.

In some aspects, the method comprises administering to the subject the composition or liposome once a week. Optionally, the DMG is diffuse intrinsic pontine glioma (DIPG) and/or the subject is age 17 years or less. In various instances, the method further comprises administering to the subject one or more lysosome-associated membrane proteins (LAMPs), as further described herein.

### BRIEF DESCRIPTION OF THE DRAWINGS

FIG. 1 is a schematic of a process of designing a peptide sequence predicted to contain MHC class II epitopes. Predictive screening to determine potential binders was performed using NetMHCII 2.3. Since the maximum input for peptide length prediction is 15-amino acids. Each sequential 15-mer encompassing the peptide was tested for potential binding (mak>5.0%) for all human HLA class II alleles available on the prediction platform.

FIG. 2 is a schematic of a process of preparing RNA molecules from E. coli-amplified, linearized plasmid DNA via in vitro transcription (IVT). The chosen sequences were placed into a plasmid containing a T7 promoter, a 5′ and 3′ untranslated region (UTR) and an incorporated poly (A) tail. Plasmids containing the coding sequence (CDS) of interest were amplified in E. coli and extracted with a commercial plasmid preparation kit. Following confirmation of the sequence, plasmids were linearized and mRNA is created through 5′ cap addition and in vitro transcription. The RNA molecules are then used in RNA-NP assembly.

FIG. 3A is a schematic of a process of administering a composition of the present disclosure. FIG. 3B is a graph of the % of CD62L⁺CD44⁺ cells of CD8⁺ splenocytes obtained from mice treated with nanoparticles alone (NP alone), nanoparticles complexed with ovalbumin RNA (OVA RNA-NP) or nanoparticles complexed with RNA encoding MHC Class II epitopes of a mutant Histone3 (H3) protein comprising a K27M mutation (H3mer RNA-NP). This demonstrates expansion of the central memory population of T cells which play a key role in maintaining long-term antigen-specific immunogenicity to their respective target which may ultimately allow long-term anti-tumor immunity. FIG. 3C is a graph of the % of PD-1⁺ cells of CD8⁺ splenocytes obtained from mice treated with nanoparticles alone (NP alone) or nanoparticles complexed with RNA encoding an MHC Class II epitope of a mutant Histone3 (H3) protein comprising a K27M mutation (H3mer RNA-NP). Transient PD-1 expression is indicative of TCR-mediated activation of T cells and reinforce the encoded peptide's capacity to induce an antigen-specific lymphocyte population.

FIG. 4A is a graph of the % of IFNγ expressing cells of CD44⁺CD62L⁺CD4⁺ splenocytes in mice administered an NP encapsulated-irrelavent RNA (eGFP), immunostimulatory-OVA encoding RNA, and the H3 mutant oligomer encoding RNA once a week for 3 weeks. Splenocytes were harvested one week after the final injection. Since IFNγ release by central memory is driven by antigen recognition, these findings support the production of an antigen specific central memory compartment in H3M-NP administered mice. FIG. 4B is a graph of the % of PD-1 expressing cells of CD44⁺CD62L⁺CD4⁺ splenocytes. The potentiated expression of PD-1 in H3M-NP treated mice relative to the irrelevant-RNA encoding control recapitulates and supports the findings presented in 3C.

FIG. 5A is a graph of % of mice surviving for the number of days post tumor implantation of untreated mice, mice treated with NPs alone, or mice treated with NPs complexed with RNA encoding a strong binding MHC Class I epitope. The treatment administration schedules in 5A-5E are outlined in 3A. While a MHC class I driven immunogenic response prolongs survival it does not produce robust long-term survivors.

FIG. 5B is a graph of % of mice surviving for the number of days post tumor implantation of untreated mice or mice treated with NPs complexed with RNA encoding MHC Class I- and Class II-restricted epitopes. With the inclusion of MHC class II alongside MHC class I, a robust survivorship outcome is accomplished in tumor-mRNA-NP treated mice.

FIG. 5C is a pair of graphs demonstrating the % of antigen specific CD8⁺ splenocytes (left) or % of cells expressing IFNγ (right) of untreated mice or mice treated with RNA alone, irrelevant RNA containing NPs (CFA-OVA), or tumor relevant RNA NPs. NP encapsulated RNA encoding MHC presenting peptides drives antigen-specific immunity and leads to a functional antigen-specific lymphocyte population.

FIG. 5D is a graph of the % of CD11c+CD86+ cells of untreated mice or mice treated with GFP RNA-NPs or total tumor mRNA NPs. Total tumor mRNA-NP treated mice show greater activation of antigen presenting dendritic cells relative to NP-alone or irrelevant-NP (GFP) treated mice.

FIG. 5E is a graph of the % of CD62+CD44+CD3+ cells of untreated mice or mice treated with GFP RNA-NPs or total tumor mRNA NPs. Administration of total tumor mRNA-NPs leads to expansion of the T cell central memory compartment relative to controls.

### DETAILED DESCRIPTION

### Nucleic Acid Molecules

The present disclosure provides a nucleic acid molecule comprising a nucleotide sequence encoding at least one MHC Class II epitope of a mutant Histone 3 (H3) protein comprising a K27M mutation. By “nucleic acid molecule” as used herein includes “polynucleotide” and “oligonucleotide” and generally means a polymer of deoxyribonucleic acid (DNA) or ribonucleic acid (RNA), which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage. In exemplary aspects, the nucleic acid molecule comprises one or more modified nucleotides, such as, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridme, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N -substituted adenine, 7-methylguanine, 5-methylammomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouratil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. In exemplary aspects, the nucleic acid molecule comprises one or more non-natural or altered inter-nucleotide linkages, such as a phosphoroamidate linkage or a phosphorothioate linkage, in place of the phosphodiester linkage found between the nucleotides of a naturally-occurring DNA molecule or RNA molecule. In exemplary aspects, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In exemplary embodiments, the nucleic acid molecule is an RNA molecule and in some aspects, the RNA molecule is a mature mRNA or a processed mRNA that lacks introns. In exemplary aspects, the RNA molecule comprises a 5′ cap, a poly (A) tail, or a combination of both. The 5′ cap in various aspects comprises a 7-methylguanylate and is attached to the 5′ end of the RNA molecule via a 5′ to 5′ triphosphate linkage. In various aspects, the 5′ cap is added to the RNA molecule via a chemical addition reaction.

In exemplary embodiments, the nucleic acid molecules are constructed based on chemical synthesis and/or enzymatic ligation reactions using procedures known in the art. See, for example, Sambrook et al., supra, and Ausubel et al., supra. In various aspects, the RNA molecules are produced outside of a cell via in vitro transcription techniques. In various aspects, the RNA molecules are synthetic RNA molecules produced by in vitro transcription. Further descriptions of exemplary methods of making the RNA molecules are provided below.

The presently disclosed nucleic acid molecules (e.g., RNA molecules) comprise a nucleotide sequence encoding at least one MHC Class II epitope of a mutant Histone 3 (H3) protein comprising a K27M mutation. By “MHC Class II epitope” refers to a portion of a protein or peptide that binds to a Major Histocompatibility Complex (MHC) Class II molecule. The MHC Class II epitope in various aspects is about 10 to about 30 amino acids long, optionally about 15 to about 24 amino acids long. In various instances, the MHC Class II epitope is about 10 to about 25 amino acids long or about 10 to about 20 amino acids long or about 15 to 30 amino acids long or about 15 to 25 amino acids long. In various aspects, due to the antigen binding groove of MHC Class II molecules having two open ends, only a portion of the MHC Class II epitope actually contacts the antigen binding groove of the MHC Class II molecule, while remaining portions of the epitope do not contact the antigen binding groove.

In exemplary aspects, the MHC Class II epitope binds to a human MHC Class II molecule. In exemplary aspects, the human MHC Class II molecule is an HLA-DP, HLA-DM, HLA-DOA, HLA-DOB, HLA-DQ, and HLA-DR. The amino acid sequences of MHC Class II molecules are known in the art and are available in the NCBI Protein database as shown in Table A.

TABLE A

| Official | NCBI | NCBI mRNA | NCBI Protein |
|---|---|---|---|
| Symbol | Gene ID | Accession No(s). | Accession No(s). |
| HLA-DMA | 3108 | NM_006120.3 | NP_006111.2 |
| HLA-DMB | 3109 | NM_002118.4 | NP_002109.2 |
| HLA-DOA | 3111 | NM_002119.3 | NP_002110.1 |
| HLA-DOB | 3112 | NM_002120.3 | NP_002111.1 |
| HLA-DPA1 | 3113 | NM_00124524.1 | NP_001229453.1 |
| | | NM_0012452525.1 | NP_001229454.1 |
| | | NM_033554.3 | NP_291032.2 |
| HLA-DPB1 | 3115 | NM_002121.5 | NP_002112.3 |
| HLA-DQA1 | 3117 | NM_002122.3 | NP_002113.2 |
| HLA-DQA2 | 3118 | NM_020056.4 | NP_064440.1 |
| HLA-DQB1 | 3119 | NM_001243961.1 | NP_001230890.1 |
| | | NM_001243962.1 | NP_001230891.1 |
| | | NM_002123.4 | NP_002114.3 |
| HLA-DQB2 | 3120 | NM_001198858.1 | NP_001185787.1 |
| | | NM_001300790.1 | NP_001287719.1 |
| HLA-DRA | 3122 | NM_019111.4 | NP_061984.2 |
| HLA-DRB1 | 3123 | NM_01243965.1 | NP_001230894.1 |
| | | NM_002124.3 | NP_002115.2 |
| HLA-DRB3 | 3125 | NM_022555.3 | NP_072049.2 |
| HLA-DRB4 | 3126 | NM_021983.4 | NP_068818.4 |
| HLA-DRB5 | 3127 | NM_002125.3 | NP_002116.2 |

The mutant H3 protein in various instances is the H3.3 protein encoded by the H3F3A gene comprising a Lys to Met mutation at amino acid position 27 or is the H3.1 protein encoded by the HIST1H3B gene comprising a Lys to Met mutation at amino acid position 27. SEQ ID NO: 1 provides the amino acid sequence of the H3.3 protein comprising the K27M mutation. In exemplary aspects, the MHC Class II epitope comprises at least 5-10 consecutive amino acids of MAPRKQLATKAARMSAPSTGGVKKPH (SEQ ID NO: 13) or RKQLATKAARMSAPSTGGVKK (SEQ ID NO: 15). In various instances, the MHC Class II epitope comprises at least 11-17 consecutive amino acids of the sequence of SEQ ID NO: 13 or SEQ ID NO: 15. In certain instances, the MHC Class II epitope comprises at least 18-21 consecutive amino acids of the sequence of SEQ ID NO: 13 or SEQ ID NO: 15. In exemplary aspects, the MHC Class II epitope comprises at least 22-25 consecutive amino acids of the sequence of SEQ ID NO: 13 or SEQ ID NO: 15. In certain instances, the MHC Class II epitope comprises the sequence of SEQ ID NO: 13 or SEQ ID NO: 15. With regard to the RNA molecules of the presently disclosed liposomes, the MHC Class II epitope comprises the sequence of ARM, RMS, or MSA, optionally, wherein the MHC Class II epitope comprises the sequence of MSAPS (SEQ ID NO: 16), RMSAP (SEQ ID NO: 17), ARMSA (SEQ ID NO: 18), AARMS (SEQ ID NO: 19), or KAARM (SEQ ID NO: 20). In exemplary instances, the RNA molecules comprise at least a portion of the nucleotide sequence of SEQ ID NO: 12 which encodes the MHC Class II epitope. In some aspects, the RNA molecules comprises the nucleotide sequence of SEQ ID NO: 14 or SEQ ID NO: 21. In exemplary aspects, the RNA molecules comprise the nucleotide sequence of SEQ ID NO: 12.

In various instances, the presently disclosed nucleic acid molecules (e.g., RNA molecules) comprise a nucleotide sequence encoding at least 2, at least 3, at least 4, or at least 5 MHC Class II epitopes of a mutant Histone 3 (H3) protein comprising a K27M mutation. In exemplary instances, the presently disclosed nucleic acid molecules (e.g., RNA molecules) comprise a nucleotide sequence encoding at least 6, at least 7, at least 8, at least 9, or at least 10 MHC Class II epitopes of a mutant Histone 3 (H3) protein comprising a K27M mutation. In exemplary aspects, the presently disclosed nucleic acid molecules (e.g., RNA molecules) comprise a nucleotide sequence encoding 10 or more MHC Class II epitopes, e.g., 11, 12, 13, 14, 15, 16, 17, 18, 19, or 20 MHC Class II epitopes, of a mutant Histone 3 (H3) protein comprising a K27M mutation.

In various aspects, the presently disclosed nucleic acid molecules (e.g., RNA molecules) comprise a nucleotide sequence encoding at least one MHC Class II epitope of a mutant Histone 3 (H3) protein comprising a K27M mutation and at least one MHC Class I epitope of the mutant H3 protein comprising the K27M mutation. By “MHC Class I epitope” refers to a portion of a protein or peptide that binds to a Major Histocompatibility Complex (MHC) Class I molecule. In exemplary aspects, the MHC Class I epitope binds to a human MHC Class I molecule. In exemplary aspects, the human MHC Class I molecule is an HLA-A, HLA-B, HLA-C, HLA-E, HLA-F, and HLA-G. The MHC Class I epitope in various aspects is about 6 to about 12 amino acids long, optionally about 8 to about 12 amino acids long. In various instances, the MHC Class I epitope is about 6 to about 10 amino acids long or about 6 to about 8 amino acids long or about 8 to 12 amino acids long or about 10 to 12 amino acids long. In exemplary aspects, the MHC Class I epitope comprises at least 6, 7, 8, 9, 10, 11, or 12 consecutive amino acids of MAPRKQLATKAARMSAPSTGGVKKPH (SEQ ID NO: 13) or RKQLATKAARMSAPSTGGVKK (SEQ ID NO: 15). With regard to the RNA molecules of the presently disclosed liposomes, the MHC Class I epitope comprises the sequence of ARM, RMS, or MSA, optionally, wherein the MHC Class II epitope comprises the sequence of MSAPS (SEQ ID NO: 16), RMSAP (SEQ ID NO: 17), ARMSA (SEQ ID NO: 18), AARMS (SEQ ID NO: 19), or KAARM (SEQ ID NO: 20). In various aspects, the RNA comprising a sequence encoding an amino acid sequence of any one SEQ ID NOs: 28-35 or any one of SEQ ID NOs: 39-42. In exemplary instances, the RNA molecules comprise at least a portion of the nucleotide sequence of SEQ ID NO: 12 which encodes the MHC Class I epitope. In some aspects, the RNA molecules comprises the nucleotide sequence of SEQ ID NO: 14 or SEQ ID NO: 21. In exemplary aspects, the RNA molecules comprise the nucleotide sequence of SEQ ID NO: 12. In various aspects, the presently disclosed nucleic acid molecules (e.g., RNA molecules) comprise a nucleotide sequence encoding at least one (e.g., at least 2, at least 3, at least 4, at least 5, at least 6, at least 7, at least 8, at least 9, at least 10, or more) MHC Class II epitope(s) and at least one (e.g., at least 2, at least 3, at least 4, at least 5, at least 6, at least 7, at least 8, at least 9, at least 10, or more) MHC Class I epitope of the mutant H3 protein comprising the K27M mutation. In various aspects, the presently disclosed nucleic acid molecules (e.g., RNA molecules) comprise a nucleotide sequence encoding multiple MHC Class II epitopes and multiple MHC Class I epitopes. In exemplary aspects, the RNA molecule encodes a peptide that binds to at least 2, at least 3, at least 4, at least 5, at least 6, at least 7, at least 8, at least 9, at least 10, at least 11, at least 12, at least 13, at least 14, at least 15, or more different HLA molecules. In exemplary aspects, the RNA molecule encodes a peptide that binds to at least 2, at least 3, at least 4, at least 5, at least 6, at least 7, at least 8, at least 9, at least 10, at least 11, at least 12, at least 13, at least 14, at least 15, or more different human Class II molecules, and/or at least 2, at least 3, at least 4, at least 5, at least 6, at least 7, at least 8, at least 9, at least 10, at least 11, at least 12, at least 13, at least 14, at least 15, or more different human Class I molecules. With being bound to a particular theory, the RNA encoding multiple MHC Class I and Class II epitopes advantageously allow it to be used in many different subjects to elicit an immune response against the mutant H3 protein.

In various instances, the presently disclosed nucleic acid molecules (e.g., RNA molecules) comprise a nucleotide sequence encoding at least one MHC Class II epitope of a mutant Histone 3 (H3) protein comprising a K27M mutation, optionally, at least one MHC Class I epitope of the mutant H3 protein, and another nucleotide sequence. In various aspects, the other nucleotide sequence encodes another epitope or another polypeptide. In exemplary aspects, the RNA molecule further comprises a nucleotide sequence encoding a lysosome-associated membrane protein (LAMP). LAMPs are membrane proteins specific to lysoomes comprising homologous lysosome-luminal domains separated by a proline-rich hinge region, a transmembrane domain and a cytoplasmic domain. A review on LAMPs is provided at Schwake et al., Traffic (2013) http://kol.org/10.1111/tra 12056. In various aspects, the nucleic acid molecule (e.g., RNA molecule) comprising a nucleotide sequence encoding a chimeric protein comprising a LAMP protein and the MHC Class II epitope of the mutant H3 protein comprising a K27M mutation. In various aspects, the LAMP protein is located N-terminal to the MHC Class II epitope. In certain aspects, the LAMP protein is a LAMP1, LAMP 2, LAMP3, LAMP4, or LAMP5 protein. The sequences of such LAMP proteins are known in the art. For example, the mRNA sequence of the LAMP1 precursor is available as NCBI Accession No. NM_005561.4 and the amino acid sequence of LAMP1 precursor is available as NCBI Accession No. NP_005552.3. Also, for example, the mRNA sequence of the LAMP2 isoform C precursor is available as NCBI Accession No. NM_001122606.1 and the amino acid sequence of LAMP2 isoform C precursor is available as NCBI Accession No. NP_001116078.1. The mRNA sequence of the LAMP3 precursor is available as NCBI Accession No. NM_014398.4 and the amino acid sequence of LAMP3 precursor is available as NCBI Accession No. NP_055213.2.

In various aspects, the nucleic acid molecules are complexed with a cationic lipid to make a particle (e.g., nanoparticle) or liposome (e.g., nanoliposome). In various instances, the RNA molecules are complexed with the cationic lipid via electrostatic interactions. In exemplary aspects, the liposomes are prepared by mixing the RNA molecules and the cationic lipid at a RNA: cationic lipid ratio of about 1 to about 10 to about 1 to about 20, optionally, about 1 to about 15. Methods of making such particles and liposomes are provided herein. Compositions, e.g., pharmaceutical compositions, comprising the nucleic acid molecule (e.g., RNA molecule) or particle or liposome are provided herein. Further descriptions of the compositions are provided below.

Additionally provided by the present disclosure are nucleic acid molecules comprising a nucleotide sequence encoding the RNA molecule of the present disclosure. In exemplary aspects, the nucleic acid molecule comprises a nucleotide sequence encoding an RNA molecule comprising a nucleotide sequence encoding at least one MHC Class II epitope of a mutant Histone 3 (H3) protein comprising a K27M mutation and optionally at least one MHC Class I epitope of the mutant H3 protein. In exemplary aspects, the nucleic acid comprises DNA and the nucleic acid molecules comprise DNA encoding the RNA molecule encoding an MHC Class II epitope of a mutant Histone 3 (H3) protein comprising a K27M mutation, optionally, the nucleic acid molecules comprise DNA comprising a sequence of SEQ ID NO: 11. Compositions, e.g., pharmaceutical compositions, comprising the nucleic acid molecule (e.g., DNA encoding the RNA molecule) are further provided herein.

### Liposomes

The present disclosure provides liposomes comprising ribonucleic acid (RNA) molecules and a cationic lipid, wherein the RNA molecules encode at least one MHC Class II epitope of a mutant Histone 3 (H3) protein comprising a K27M mutation, optionally, at least one MHC Class I epitope, and compositions (e.g., pharmaceutical compositions) comprising the liposomes.

Liposomes are artificially-prepared vesicles which, in exemplary aspects, are primarily composed of a lipid bilayer. Liposomes in various instances are used as a delivery vehicle for the administration of nutrients and pharmaceutical agents. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than 50 nm in diameter, and (c) a large unilamellar vesicle (LUV) which may be between 50 and 500 nm in diameter. Liposomes in various instances are designed to comprise opsonins or ligands in order to improve the attachment of liposomes to unhealthy tissue or to activate events such as, but not limited to, endocytosis. In exemplary aspects, liposomes contain a low or a high pH in order to improve the delivery of the pharmaceutical formulations. In various instances, liposomes are formulated depending on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In exemplary aspects, the liposome has a diameter within the nanometer range and accordingly in certain instances are referred to herein as “nanoliposomes” or “nanoparticles” (abbreviated as NPs). In exemplary aspects, the liposome has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, about 400 nm to about 500 nm. In exemplary aspects, the liposome has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, about 300 nm±5 nm. In exemplary aspects, the liposome is about 50 nm to about 250 nm in diameter. In some aspects, the liposome is about 70 nm to about 200 nm in diameter. In exemplary aspects, the composition comprises a heterogeneous mixture of liposomes ranging in diameter, e.g., about 50 nm to about 500 nm or about 50 nm to about 250 nm in diameter. Optionally, the composition comprises a heterogeneous mixture of liposomes ranging from about 70 nm to about 200 nm in diameter.

In exemplary aspects, the liposome has a zeta potential of about 30 mV to about 60 mV. In other words, in certain aspects, the liposome has an overall surface net charge of about 30 mV to about 60 mV (e.g., about 30 mV to about 55 mV, about 30 mV to about 50 mV, 30 mV to about 45 mV, about 30 mV to about 40 mV, about 30 mV to about 35 mV, about 35 mV to about 60 mV, about 40 mV to about 60 mV, about 45 mV to about 60 mV, about 50 mV to about 60 mV, or about 55 mV to about 60 mV. In exemplary aspects, the liposome has an overall surface net charge of about 40 mV to about 50 mV.

In exemplary embodiments, the liposomes comprise a cationic lipid. In some embodiments, the cationic lipid is a low molecular weight cationic lipid such as those described in U.S. patent application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

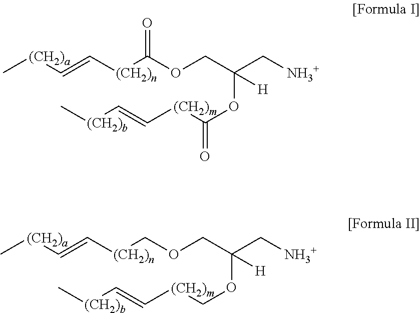

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the liposomes are formed from 1,2-dioleyloxy-N, N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety). In some embodiments, the liposomes are formed from the synthesis of stabilized plasmid-lipid particles (SPLP) or stabilized nucleic acid lipid particle (SNALP) that have been previously described and shown to be suitable for oligonucleotide delivery in vitro and in vivo. The liposomes in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In exemplary instances, the liposome comprises 48% cholesterol, 20% DSPC, 2% PEG-c-DMA, and 30% cationic lipid, where the cationic lipid can be 1,2-distearloxy-N, N-dimethylaminopropane (DSDMA), DODMA, DLin-DMA, or 1,2-dilinolenyloxy-3-dimethylaminopropane (DLenDMA), as described by Heyes et al.

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy) heptadecanedioate (L319), and further comprise a neutral lipid, a sterol and a molecule capable of reducing particle aggregation, for example a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. US20130150625, herein incorporated by reference in its entirety. As a non-limiting example, the cationic lipid in certain aspects is 2-amino-3-[(9Z,12Z)-octadeca-9, 12-dien-1-yloxy]-2-{[(9Z,2Z)-octadeca-9, 12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9, 12-dien-1-yloxy]-2-[(octyloxy)methyl]propan-1-ol (Compound 3 in US20130150625); and 2-(dimethylamino)-3-[(9Z,12Z)-octadeca-9, 12-dien-1-yloxy]-2-{[(9Z, 12Z)-octadeca-9, 12-dien-1-yloxy]methyl}propan-1-ol (Compound 4 in US20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-CDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which are herein incorporated by reference in their entirety)

In exemplary aspects, the cationic lipid may be selected from (20Z,23Z)-N,N-dimethylnonacosa-20,23-dien-10-amine, (17Z,20Z)-N,N-dimemylhexacosa-17,20-dien-9-amine, (1Z, 19Z)-N,N-dimethylpentacosa-1 6, 19-dien-8-amine, (13Z, 16Z)-N,N-dimethyldocosa-13, 16-dien-5-amine, (12Z, 15Z)-N,N-dimethylhenicosa-12, 15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z, 18Z)-N,N-dimethyltetracosa-15, 18-dien-5-amine, (14Z, 17Z)-N,N-dimethyltricosa-14, 17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z, 19Z)-N,N-dimethylpentacosa-16, 19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z, 14Z)-1-nonylicosa-11, 14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z, 15Z)-N,N-dimethyl-2-nonylhenicosa-12,15-dien-1-amine, (13Z, 16Z)-N,N-dimethyl-3-nonyldocosa-13, 16-dien-1-amine, N, N-dimethyl-1-[(1S,2R)-2-octylcyclopropyl]eptadecan-8-amine, 1-[(1S,2R)-2-hexylcyclopropyl]-N,N-dimethylnonadecan-10-amine, N, N-dimethyl-1-[(1S,2R)-2-octylcyclopropyl]nonadecan-10-amine, N,N-dimethyl-21-[(1S,2R)-2-octylcyclopropyl]henicosan-10-amine, N, N-dimethyl-1-[(1S,2S)-2-{[(1R,2R)-2-pentylcyclopropyl]methyl}cyclopropyl]nonadecan-10-amine, N, N-dimethyl-1-[(1S,2R)-2-octylcyclopropyl]hexadecan-8-amine, N, N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N, N-dimethylpentadecan-6-amine, N, N-dimethyl-1-[(1S,2R)-2-octylcyclopropyl]pentadecan-8-amine, R-N, N-dimethyl-1-[(9Z,12Z)-octadeca-9, 12-dien-1-yloxy]-3-(octyloxy) propan-2-amine, S-N,N-dimethyl-1-[(9Z,12Z)-octadeca-9, 12-dien-1-yloxy]-3-(octyloxy) propan-2-amine, 1-{2-[(9Z,12Z)-octadeca-9, 12-dien-1-yloxy]-1-[(octyloxy)methyl]ethyl}pyrrolidine, (2S)-N, N-dimethyl-1-[(9Z, 12Z)-octadeca-9, 12-dien-1-yloxy]-3-[(5Z)-oct-5-en-1-yloxy]propan-2-amine, 1-{2-[(9Z,12Z)-octadeca-9, 12-dien-1-yloxy]-1-[(octyloxy)methyl]ethyl}azetidine, (2S)-1-(hexyloxy)-N,N-dimethyl-3-[(9Z,12Z)-octadeca-9, 12-dien-1-yloxy]propan-2-amine, (2S)-1-(heptyloxy)-N,N-dimethyl-3-[(9Z, 12Z)-octadeca-9, 12-dien-1-yloxy]propan-2-amine, N,N-dimethyl-1-(nonyloxy)-3-[(9Z,12Z)-octadeca-9, 12-dien-1-yloxy]propan-2-amine, N, N-dimethyl-1-[(9Z)-octadec-9-en-1-yloxy]-3-(octyloxy) propan-2-amine; (2S)-N,N-dimethyl-1-[(6Z,9Z,12Z)-octadeca-6,9, 12-trien-1-yloxy]-3-(octyloxy) propan-2-amine, (2S)-1-[(11Z, 14Z)-icosa-11,14-dien-1-yloxy]-N,N-dimethyl-3-(pentyloxy) propan-2-amine, (2S)-1-(hexyloxy)-3-[(11Z,14Z)-icosa-11, 14-dien-1-yloxy]-N,N-dimethylpropan-2-amine, 1-[(11Z, 14Z)-icosa-11, 14-dien-1-yloxy]-N,N-dimethyl-3-(octyloxy) propan-2-amine, 1-[(13Z, 16Z)-docosa-13, 16-dien-1-yloxy]-N,N-dimethyl-3-(octyloxy) propan-2-amine, (2S)-1-[(13Z, 16Z)-docosa-13, 16-dien-1-yloxy]-3-(hexyloxy)-N,N-dimethylpropan-2-amine, (2S)-1-[(13Z)-docos-13-en-1-yloxy]-3-(hexyloxy)-N, N-dimethylpropan-2-amine, 1-[(13Z)-docos-13-en-1-yloxy]-N,N-dimethyl-3-(octyloxy) propan-2-amine, 1-[(9Z)-hexadec-9-en-1-yloxy]-N, N-dimethyl-3-(octyloxy) propan-2-amine, (2R)-N,N-dimethyl-H (1-metoyloctyl)oxyl-3-[(9Z,12Z)-octadeca-9, 12-dien-1-yloxy]propan-2-amine, (2R)-1-[(3,7-dimethyloctyl)oxy]-N,N-dimethyl-3-[(9Z,12Z)-octadeca-9, 12-dien-1-yloxy]propan-2-amine, N, N-dimethyl-1-(octyloxy)-3-({8-[(1S,2S)-2-{[(1R,2R)-2-pentylcyclopropyl]methyl}cyclopropyl]octyl}oxy) propan-2-amine, N, N-dimethyl-1-{[8-(2-oclylcyclopropyl) octyl]oxy}-3-(octyloxy) propan-2-amine and (11E,20Z,23Z)-N,N-dimethylnonacosa-11,20,2-trien-10-amine or a pharmaceutically acceptable salt or stereoisomer thereof.

In some embodiments, the composition comprises a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

### Methods of Generating Liposomes

The present disclosure further provides methods of generating a liposome comprising ribonucleic acid (RNA) molecules and a cationic lipid, wherein the RNA molecules encode at least one MHC Class II epitope (and optionally at least one MHC Class I epitope) of a mutant Histone 3 (H3) protein comprising a K27M mutation and optionally at least one MHC Class I epitope of the mutant H3 protein.

In exemplary embodiments, the method comprises mixing any of the presently disclosed RNA molecules encoding at least one MHC Class II epitope (and optionally at least one MHC Class I epitope) of a mutant H3 protein comprising a K27M mutation (and optionally, at least one MHC Class I epitope) with a cationic lipid, including any one or more of those described herein. In various aspects, the method comprises mixing the RNA molecules and the cationic lipid at a RNA: cationic lipid ratio of about 1 to about 10 to about 1 to about 20, optionally, about 1 to about 15.

In exemplary embodiments, the method comprises producing the RNA molecules and then mixing the RNA molecules with the cationic lipid. In various instances, the RNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates. In exemplary aspects, the kit is employed with a nucleic acid encoding the RNA molecule encoding the MHC Class II epitope (and optionally at least one MHC Class I epitope) of a mutant H3 protein comprising a K27M mutation and optionally the MHC Class I epitope of the mutant H3 protein. Optionally, the nucleic acid comprises the sequence of SEQ ID NO: 11. Accordingly, in exemplary aspects, the method comprises in vitro transcribing a nucleic acid comprising a nucleotide sequence encoding the RNA molecules encoding at least one MHC Class II epitope (and optionally at least one MHC Class I epitope) of a mutant H3 protein comprising a K27M mutation (and optionally at least one MHC Class I epitope) and mixing the RNA molecules with a cationic lipid. In various aspects, the nucleic acid comprises a sequence encoding a poly (A) tail so that the in vitro transcribed RNA molecule comprises a poly(A) tail at the 3′ end. The method in various aspects, comprises additional processing steps, such as, for example, capping the in vitro transcribed RNA molecules. In exemplary instances, the method of generating a liposome comprising RNA molecules and a cationic lipid comprises (i) in vitro transcribing a nucleic acid comprising a nucleotide sequence encoding an RNA molecule encoding at least one MHC Class II epitope (and optionally at least one MHC Class I epitope) of a mutant H3 protein comprising a K27M mutation, (ii) chemically adding a 5′-cap to the in vitro transcribed RNA molecules, and (iii) mixing the RNA molecules comprising the 5′-cap with a cationic lipid. In various aspects, the method comprises mixing the RNA molecules and the cationic lipid at a RNA: cationic lipid ratio of about 1 to about 10 to about 1 to about 20, optionally, about 1 to about 15. With regard to the method, the cationic lipid may be any of those described herein. In various aspects, the cationic lipid is DOTAP. In various aspects, the RNA molecules and cationic lipid are mixed at a RNA: lipid ratio of about 1 to about 10 to about 1 to about 20. Optionally, the RNA molecules and cationic lipid are mixed at a RNA: lipid ratio of about 1 to about 15. In various aspects, the nucleotide sequence encoding the RNA molecules comprises the sequence of SEQ ID NO: 11. In various instances, the nucleotide sequence encoding the RNA molecules is operably linked to a promoter, optionally a T7 promoter. The nucleotide sequence encoding the RNA molecules is flanked by a 5′ untranslated region (5′UTR) and a 3′ untranslated region (3′UTR). In various aspects, the nucleic acid comprises a sequence encoding a polyA tail. In exemplary instances, the nucleic acid is a linearized plasmid.

In exemplary aspects, the method comprises downstream steps to prepare the liposomes for administration to a subject, e.g., a human. In exemplary instances, the method comprises formulating the lipid for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

The present disclosure also provides the liposome(s) generated by these presently disclosed methods. The liposome in some aspects may be formulated for intravenous injection.

### Compositions

The present disclosure provides compositions relating to the presently disclosed nucleic acid molecules and liposomes. In exemplary embodiments, the composition comprises a liposome comprising ribonucleic acid (RNA) molecules and a cationic lipid, wherein the RNA molecules encode an MHC Class II epitope (and optionally at least one MHC Class I epitope) of a mutant Histone 3 (H3) protein comprising a K27M mutation. The compositions comprise any of the liposomes described herein. See, e.g., the section entitled Liposomes. For instance, the composition in exemplary aspects comprises a homogeneous population of a single type of liposome described herein. In alternative aspects, the composition comprises a heterogeneous mixture of liposomes that vary in size, zeta potential, amount of cationic lipid, amount of nucleic acid molecules, type of cationic lipid, and/or type of nucleic acid molecules. In exemplary aspects, the composition comprises about 10¹⁰ liposomes per mL to about 10¹⁵ liposomes per mL (e.g, about 10¹⁰ liposomes per mL, about 10¹¹ liposomes per mL, about 10¹² liposomes per mL, about 10¹³ liposomes per mL, about 10¹⁴ liposomes per mL. In some aspects, the composition comprises about 10¹² liposomes+10% per mL. In exemplary aspects, the composition is administered in an amount based on the weight of the subject. In exemplary aspects, about 1 to about 10 μL (e.g., about 2 to about 7 μL, about 2, 3, 4, 5, 6, o r7 μL, about 2.5 μL) of a solution comprising about 10¹² liposomes per mL is administered per kg body weight.

In various aspects, the compositions of the present disclosure comprises components in addition to the liposome, nucleic acid molecule encoding the MHC Class II epitope (and optionally at least one MHC Class I epitope) and/or nucleic acid molecule comprising a sequence encoding the nucleic acid molecule encoding the MHC Class II epitope (and optionally at least one MHC Class I epitope). In some aspects, the compositions further comprise a pharmaceutically acceptable carrier, excipient or diluent. In exemplary aspects, the composition is a pharmaceutical composition intended for administration to a human. In exemplary aspects, the composition is a sterile composition. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration. In exemplary aspects, when the composition comprises cells comprising the liposomes (and not liposomes outside of cells), the composition is suitable for intradermal administration. In exemplary aspects, the composition is systemically administered via parenteral administration. In exemplary aspects, the composition is administered via injection or infusion. In exemplary instances, the composition is administered subcutaneously or intravenously or intramuscularly. In some aspects, the composition is administered intravenously.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

In some embodiments, the composition of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the liposome composition may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

### Methods of Use

Without being bound to any particular theory, the data provided herein for the first time support the use of the liposomes comprising RNA molecules and a cationic lipid, wherein the RNA molecules encode an MHC Class II epitope (and optionally at least one MHC Class I epitope) of a mutant Histone 3 (H3) protein comprising a K27M mutation, in methods of increasing in a subject the level or number of central memory T cells. In various aspects, the liposomes are particularly useful for increasing in a subject the level or number of central memory T cells having antigen specificity for an epitope of a mutant Histone 3 (H3) protein comprising a K27M mutation. In various aspects, the liposomes are particularly useful for increasing in a subject the level or number of central memory T cells which activate the immune system against tumor cells, e.g., cells of a diffuse midline glioma (DMG). Accordingly, the present disclosure provides methods of increasing in a subject the number of central memory T cells having antigen specificity for an epitope of a mutant Histone 3 (H3) protein comprising a K27M mutation. In exemplary aspects, the method comprises administering to the subject a liposome comprising RNA molecules and a cationic lipid, wherein the RNA molecules encode an MHC Class II epitope (and optionally at least one MHC Class I epitope) of a mutant Histone 3 (H3) protein comprising a K27M mutation, or a composition comprising the same, in an amount effective to increase the central memory T cells in the subject. The present disclosure also provides methods of enhancing in a subject an immune response against tumor cells, e.g., cells of a DMG, expressing a mutant Histone 3 (H3) protein comprising a K27M mutation. In exemplary aspects, the method comprises administering to the subject a liposome comprising RNA molecules and a cationic lipid, wherein the RNA molecules encode an MHC Class II epitope (and optionally at least one MHC Class I epitope) of a mutant Histone 3 (H3) protein comprising a K27M mutation, or a composition comprising the same, in an amount effective to increase the an immune response against the DMG in the subject. In exemplary aspects, the subject has a diffuse midline glioma (DMG) expressing a mutant Histone 3 (H3) protein comprising a K27M mutation or a predisposition to having the DMG. The subject in various instances has been treated for a DMG or is in remission for the DMG. Optionally, the method further comprises administering to the subject one or more lysosome-associated membrane proteins (LAMPs).

As used herein, the term “increase” and “enhance” and words stemming therefrom may not be a 100% or complete increase or enhancement. Rather, there are varying degrees of increasing or enhancing of which one of ordinary skill in the art recognizes as having a potential benefit or therapeutic effect. The presently disclosed methods may for instance delay the onset or re-occurrence/relapse of the disease or a symptom thereof to any amount or level. In exemplary embodiments, the increase or enhancement provided by the methods is at least or about a 10% increase or enhancement (e.g., at least or about a 20% increase or enhancement, at least or about a 30% increase or enhancement, at least or about a 40% increase or enhancement, at least or about a 50% increase or enhancement, at least or about a 60% increase or enhancement, at least or about a 70% increase or enhancement, at least or about a 80% increase or enhancement, at least or about a 90% increase or enhancement, at least or about a 95% increase or enhancement, at least or about a 98% increase or enhancement).

Methods of treating a subject with a diffuse midline glioma (DMG) expressing a mutant Histone 3 (H3) protein comprising a K27M mutation are furthermore provided herein. In exemplary aspects, the method comprises administering to the subject a liposome comprising RNA molecules and a cationic lipid, wherein the RNA molecules encode an MHC Class II epitope (and optionally at least one MHC Class I epitope) of a mutant Histone 3 (H3) protein comprising a K27M mutation, or a composition comprising the same, in an amount effective to treat the DMG in the subject.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease, or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

With regard to the foregoing methods, the liposomes or the composition comprising the same in some aspects is systemically administered to the subject. Optionally, the method comprises administration of the liposomes or composition by way of parenteral administration. In various instances, the liposome or composition is administered to the subject intravenously.

In various aspects, the liposome or composition is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various aspects, the liposomes or composition is/are administered to the subject once a week.

### Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less. In exemplary aspects, the subject has a DMG. In various instances, the DMG is diffuse intrinsic pontine glioma (DIPG).

The following examples are given merely to illustrate the present invention and not in any way to limit its scope.

### EXAMPLES

### Example 1

This example describes a method of generating a liposome of the present disclosure.

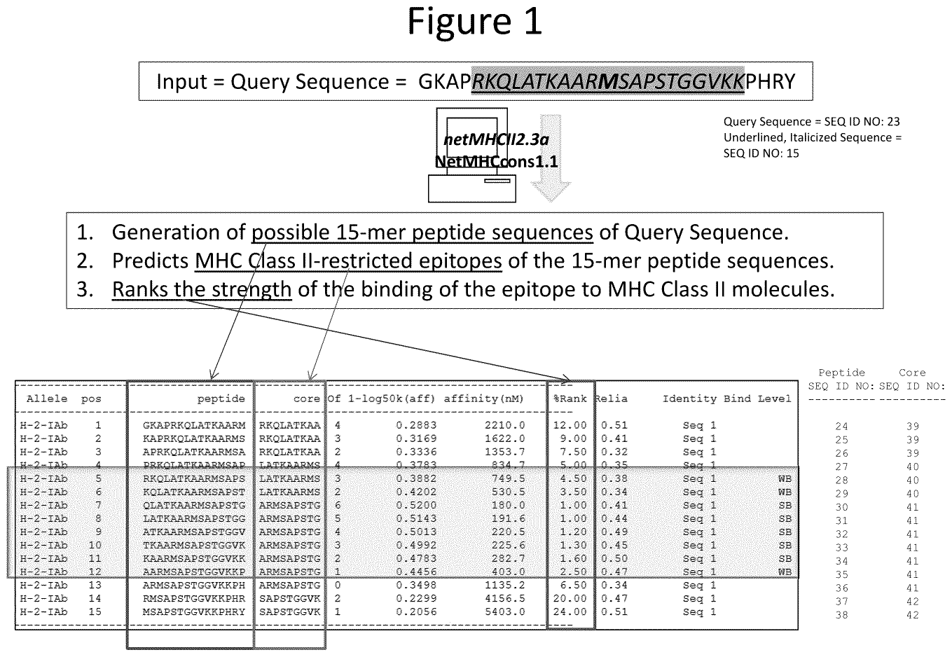

An MHC Class II epitope of a mutant Histone 3 (H3) protein comprising a K27M mutation was selected by using the NetMHCII2.3a and NetMHCcons1.1 prediction servers to predict potential MHC-restricted epitopes present within a peptide sequence encompassing the K27M mutation of the mutant H3 protein. For a given input amino acid sequence, the netMHCII2.3a server predicts the MHC Class II binding site(s) across multiple mouse and human MHCII alleles within a sequence of up to 15 amino acids of the input sequence (query sequence). The alleles included a range of human HLA Class II subtypes including HLA-DR (7/25), HLA-DPA (2/9), HLA-DQA (10/20), and HLA-A and B* (10/12). The current algorithm is available at the IP address “http://” followed by “www.cbs.dtu.dk/services/NetMHCII/”. FIG. 1 shows the mutant 26-mer (25-mer including start codon) query sequence chopped up into 15-mer segments for prediction and the affinity and % rank for each 15-mer segment. The table at the bottom of FIG. 1 lists amino acid sequences reflecting the range of 15-mers that possess weak to strong binding. Potential strong binders are displayed as having a % rank<5.0. As you can see from FIG. 1, the sequence RKQLATKAARMSAPSTGGVKKP (SEQ ID NO: 22) contained 15-mers with a % rank of less than 5.0. A sequence comprising SEQ ID NO: 22 (H3 Mut 26-mer) was identified through this analysis.

The above-described in silico analysis was also performed on an input wild-type (WT) H3 sequence lacking the K27M mutation. Through this analysis, a correlative WT 26-mer sequence comprising predicted MHC Class II strong binding epitopes was identified.

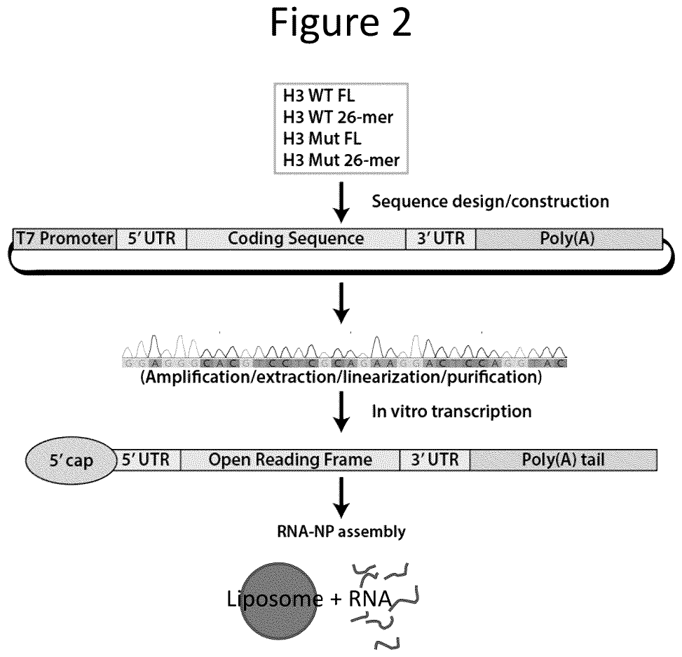

DNA template molecules encoding a H3 Mut 26-mer or the version lacking the K27M mutation (H3 WT 26-mer), or encoding full length versions of the mutant H3 protein (H3 Mut FL) or WT H3 protein (H3 WT FL) were synthesized. Each DNA template molecule was engineered into a plasmid comprising a T7 promoter, 5′- and 3′-UTRs flanking the DNA template molecules (coding sequence), and a sequence encoding a poly (A) tail. A visual of the sequence design of the plasmid is shown in FIG. 2. Plasmids comprising the different DNA template molecules were amplified, extracted, then linearized and purified. The linearized, purified plasmids were then used as templates for RNA production via in vitro transcription (IVT). IVT was carried out using the mMESSAGE mMACHINE™ T7 transcription kit (ThermoFisher Scientific, Catalog number AM1344). The resulting RNA molecules comprising a poly (A) tail were used in a chemical capping reaction to add a cap at the 5′ end of the RNA molecule. These capped RNA molecules were subsequently used to assemble RNA nanoparticles (NP) or liposomes.

FIG. 2 provides an exemplary schematic of the steps that result in the generation of RNA NPs. Nanoparticles (NPs), also referred to herein as liposomes, may be generated by any means known in the art, including but not limited to the methods described in Sayour et al., Oncoimmunology 2016, e1256527. In Sayour et al., 2016, supra, it is taught that the cationic lipid DOTAP (powder form) is acquired from Avanti, Polar Lipids Inc. (Alabaster, AL, USA). For preparation, chloroform is added to re-suspend 25-100 mg; chloroform is evaporated off until a thin lipid layer remains. The mixture is re-suspended in 5-20 mL of PBS before being placed in 50° C. water bath for 1-2 hours with intermittent vortexing. Within twenty-four hours, PBS (5-20 mL) is added to the mixture, vortexed and placed in a bath sonicator for 5 minutes before passage through a 0.43 μm and a 0.22 μm syringe filter (PALL Acrodisc syringe filter with Supor membrane). The final NP solution (2.5 μg/μL) is based on pre-filtration DOTAP concentration (2.5 μg/μL).

For in vivo studies: 25 μg of RNA is added to 375 μg of DOTAP (per mouse) in PBS buffer. For in vitro studies: 1.67 μg of RNA is added to 25 μg of DOTAP (per 1×10⁵ cells) in PBS buffer. In both cases, the mixture is kept at room temperature (~15-20 minutes) to facilitate complex formation.

### Example 2

This example describes an in vivo method of administering the liposomes to evaluate the immunogenicity of the liposomes designed to target the mutant H3 protein comprising the K2M mutation.

The purpose of this study was to show that H3.3 K27M neoantigens can drive a immunotherapeutic response against DIPG via RNA-NPs (liposomes). Neoantigen targeted RNA-NPs were formulated and examined for their immunogenicity using CD4+ and CD8+ T cell proliferation and cytotoxicity assays.

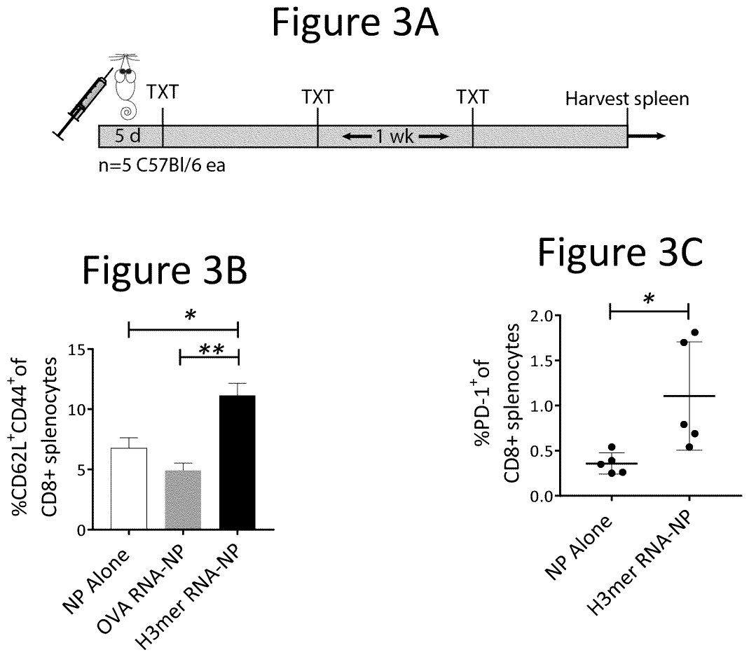

C57BL/6 mice, and Tdtomato reporter A14 mice and OT-I transgenic mice on the C57BL/6 background were purchased from Jackson Laboratories. Animal procedures were approved by the University of Florida Institutional Animal Care and Use Committee. Mice (n=5) were treated with (A) RNA-NPs comprising capped RNA molecules encoding a H3 Mut 26-mer described in Example 1 (H3mer RNA-NP), (B) NPs comprising ovalbumin RNA (OVA RNA-NP or OVA-NP) which served as a positive control, or (C) NPs without any RNA (a negative control; NP alone). Nanoparticles (200 μL) were injected into the tail vein of C57BI/6 mice. Mice were treated once a week for 3 weeks. Mice were sacrificed one week following the last treatment and spleens were harvested. A schematic of the treatment protocol is shown in FIG. 3A.

Splenocytes of the harvested spleens were phenotypically analyzed by flow cytometry. As shown in FIG. 3B, the % of CD62+CD44+ cells of CD8+ splenocytes were increased in the spleens of mice treated with H3mer RNA-NP, compared to controls, suggesting that the central memory phenotype in H3mer-NP administered mice was increased. The % of PD-1⁺ cells of CD8+ splenocytes were also measured. As shown in FIG. 3C, mice treated with H3mer RNA-NP exhibited an elevated expression of PD-1 in CD8⁺ T cells, suggesting that T cell activation was induced. These data demonstrate that mice treated with H3-mer RNA NPs increased activated central memory T cells.

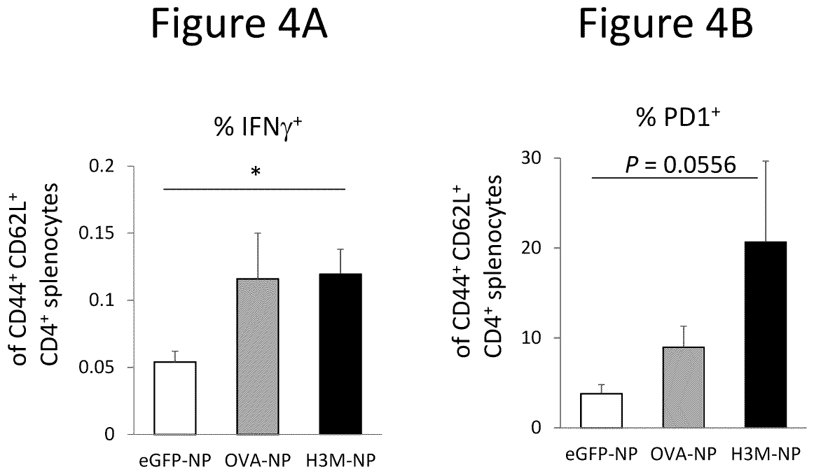

The expression of IFNγ and PD1+ by CD44+CD62+ cells of the CD4+ subset of splenocytes harvested from mice was measured by flow cytometry. As shown in FIG. 4A, IFNγ expression of CD44+CD62+ cells of the CD4+ subset of splenocytes was increased in the spleens of H3M-NP treated mice. PD1 expression by these cells was likewise increased (FIG. 4B). These data suggest that CD4 memory cell expression of IFNγ and PD1 is elevated in mice treated with the H3mer-RNA NPs.

The above results are consistent with previous observations made in separate experiments.

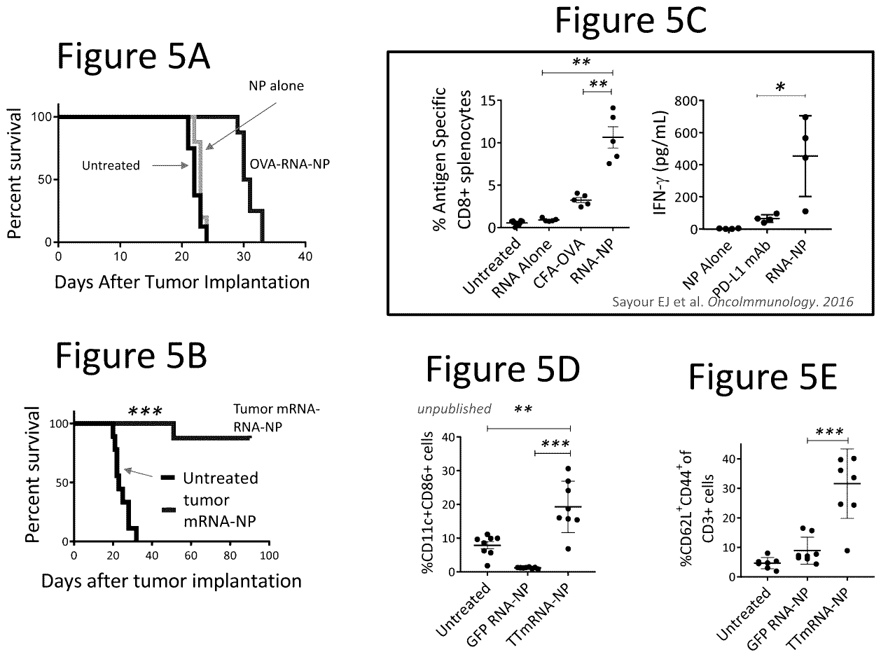

In a first separate experiment, mouse models expressing highly immunogenic OVA peptide were injected with OVA-RNA NPs, NP alone, or untreated. In this experiment, OVA-RNA NPs represent NPs comprising RNA molecules encoding MHC Class I-restricted epitopes of a tumor antigen. The % of mice surviving for the indicated number of days post tumor implantation were observed and recorded. As shown in FIG. 5A, the % survival past 30 days post tumor implantation was increased in mice treated with OVA-RNA NPs, compared to untreated mice and mice treated with NP alone (without RNA).

In another separate experiment, mouse models of another type were injected with NPs complexed with total tumor RNA (tumor mRNA-NP) or left untreated. In this experiment, tumor mRNA NPs represent NPs comprising RNA molecules encoding MHC Class I- and MHC Class II epitopes of a tumor antigen. The % of mice surviving for the indicated number of days post tumor implantation were observed and recorded. As shown in FIG. 5B, the mice treated with total tumor RNA NPs survived beyond 100 days, whereas untreated mice did not survive past 40 days.

Taken together these data suggest the importance of NPs comprising RNA molecules encoding both Class I and Class II-restricted epitopes.

In previous studies, it was shown that the % of antigen specific CD8+ spenocytes of RNA-NP treated mice was increased compared to control mice treated with RNA alone, NPs complexed with control RNA, or untreated. Also, our previous work shows that the such cells demonstrated higher IFNγ expression, which confirms the generation of antigen-specific immunogenicity. These data support that antigen specific CD8+ cells are activated upon treatment with NPs complexed with tumor relevant RNA molecules. FIG. 5C. A similar effect was observed in a separate experiment wherein mice were treated with NPs complexed with total tumor RNA (TTmRNA-NP). Mice treated with TTmRNA-NP displayed a higher percentage of CD11c+CD86+ cells and also a higher percentage of CD62L+CD44+CD3+ cells, relative to untreated mice and mice treated with Green Fluorescence Protein (GFP) RNA complexed NPs (FIGS. 5D and 5E). The observations shown in FIGS. 5A-5E collectively support that Class I restricted RNA epitopes do not mediate survivor benefits but total tumor RNA antigens mediate substantial survivorship.

This example demonstrates the importance of MHC Class II epitopes of a mutant H3 protein.

### Example 3

This example describes a method of using LAMP vaccine in combination with the NPs of Example 1.

RNA molecules encoding a chimeric protein comprising LAMP and the MHC Class II epitope of the mutant H3 protein comprising the K27M mutation are made by in vitro translation as essentially described in Example 1. These RNA molecules are complexed with nanoparticles (liposomes). The formulated nanoparticles are then injected into the tail vein of mice as essentially described in Example 2. CTL assays using the splenocytes from harvested spleens are carried out as described in Example 2, as well as the measurements of % effector memory, % central memory and % PD-1 expressing cells.

Also, we will assay for enhanced MHC II presentation in LAMP and DC-LAMP conjugated templates. Tumor bearing, eYFP reporting IFNγ, “GREAT,” mice will be administered 1) NPs alone, or: 2) neoantigen, 3) LAMP/neoantigen, 4) DC-LAMP/neoantigen, 5) equimolar LAMP and DC-LAMP/neoantigen RNA-NPs (n=10 per group; see Aim 1 admin.). DIPG cell line with hallmark mutations is courtesy Oren Becher, MD, at Northwestern. One week later we will harvest spleens and tumors for tetramer, Th1/Th2, DC, and T cell flow cytometry. If LAMPs enhance MHC II presentation, we anticipate increased CD4+ and CD8+ T cell recruitment as well as elevated IFNγ⁺ and tetramer⁺ cells

Without being bound to a particular theory, the inclusion of the RNA encoding the LAMP leverages a trafficking signal which guides the antigen (H3K27M) to the lysosomes in antigen presenting cells. It is compelled to enter a pathway that results in the presentation of the antigen to CD4+ cells, often referred to as “helper T cells”. CD4+ T cells are involved in affecting many aspects of a complex immune response: cytokine release, immunological memory, antibody production, and support for other T cell types. It is also believed that the inclusion of LAMP RNA will drive the orientation of the CD4+ T cell to a particular sub-type, the Th1 T cell. Th1 cells produce inflammatory and immunity inducing chemical messengers, cytokines, that can help stimulate the immune response. This approach is also believed to induce a broad immune response and thereby results in enhanced CD8+ T cell responses as well.

### Example 4

This example demonstrates the immunologic targeting of DIPG with H3K27M encoding RNA-nanoparticles.

Background: DIPG remains uniformly recalcitrant and necessities development of novel targeted therapies. The histone mutation in H3K27M is conserved in the preponderance of DIPG patients and may be exploited as a neoepitope for cancer immunotherapy. We have developed a novel treatment platform, which leverages the use of clinically translatable nanoparticles (NPs) combined with H3K27M mRNA neoantigens for in vivo activation of dendritic cells and generation of DIPG specific T cells.

Objective: Since neoantigens have been shown to mediate immunologic response through MHC class II, we sought to identify MHCII restricted epitopes spanning the H3K27M junction for development of RNA-NP vaccines against DIPG

Results: We identified a MHC Class II-restricted H3K27M epitope that was not present in the wild-type sequence. We constructed a 25-mer sequence (75 amino acids) spanning this range, before cloning it into our custom pGEM-4z plasmid containing a poly A tail and 5′ UTR with a T7 promoter for amplification of MHC-II restricted H3K27M mRNA. We complexed this RNA with our custom NP formulation and investigated its immunogenicity in C57BI/6 mice. Unlike MHC-I restricted epitopes (i.e. OVAlbumin's SINFEKL epitope) MHC-II restricted H3K27M epitopes elicited significant increases in activated CD4 and CD8 central memory T cells. We demonstrate that this central memory phenotype correlates with anti-tumor efficacy and long-term survival outcomes in murine tumor models.

Conclusion: RNA-NPs can be made readily available for all DIPG patients (and not only HLA specific haplotypes) providing a renewable antigen resource that can be used to continuously vaccinate patients for months/years after diagnosis. Herein, we identify MHCII restricted K27M epitopes for activation of immunologic central memory necessary for long-lived anti-tumor efficacy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1-59. (canceled)

60. A composition comprising a liposome comprising ribonucleic acid (RNA) molecules and a cationic lipid, wherein the RNA molecules encode at least one MHC Class II epitope (i) comprising at least 30 amino acids of a mutant Histone 3 (H3) protein comprising a K27M mutation and (ii) comprising an amino acid sequence of SEQ ID NO: 24, wherein the liposome has a zeta potential of about 30 mV to about 60 mV.

61. The composition of claim 60, wherein the RNA molecules encode at least 2 MHC Class II epitope of the mutant H3 protein.

62. The composition of claim 60, wherein the RNA molecules encode a peptide that binds to at least one or more human MHC Class II molecules.

63. The composition of claim 60, wherein the liposome comprises RNA molecules that encode at least one MHC Class I epitope of the mutant H3 protein.

64. The composition of claim 60, wherein the RNA molecules encode a peptide that binds to at least one or more human MHC Class I molecules.

65. The composition of claim 60, wherein the cationic liposome is 1,2-dioleoyl-3-trimethylammonium-propane (DOTAP).

66. The composition of claim 60, wherein the composition comprises a plurality of liposomes, each liposome of which is about 50 nm to about 250 nm in diameter.

67. The composition of claim 60, wherein the RNA molecules are complexed with the cationic lipid via electrostatic interactions.

68. The composition of claim 60, wherein the RNA molecules comprise a 5′-cap.

69. The composition of claim 60, wherein the RNA molecules further encode a lysosome-associated membrane protein (LAMP).

70. A method of generating a liposome comprising ribonucleic acid (RNA) molecules and a cationic lipid, wherein the RNA molecules encode at least one MHC application Ser. No. 19/434,197 Class II epitope comprising at least 30 amino acids of a mutant Histone 3 (H3) protein comprising a K27M mutation and comprising an amino acid sequence of SEQ ID NO: 24, said method comprising (i) in vitro transcribing a nucleic acid comprising a nucleotide sequence encoding the RNA molecules, (ii) chemically adding a 5′-cap to the in vitro transcribed RNA molecules, and (iii) mixing the RNA molecules comprising the 5′-cap with a cationic lipid, wherein the liposome has a zeta potential of about 30 mV to about 60 mV.

71. A liposome generated by the method of claim 70.

72. A composition comprising the liposome of claim 60.

73. A method of increasing in a subject the number of central memory T cells having antigen specificity for an epitope of a mutant Histone 3 (H3) protein comprising a K27M mutation, said method comprising administering to the subject a composition of claim 60 in an amount effective to increase the central memory T cells in the subject.

74. A method of enhancing in a subject an immune response against a diffuse midline glioma (DMG) expressing a mutant Histone 3 (H3) protein comprising a K27M mutation, said method comprising administering to the subject a composition of claim 60 in an amount effective to increase an immune response against the DMG in the subject.

75. A method of treating a subject with a diffuse midline glioma (DMG) expressing a mutant Histone 3 (H3) protein comprising a K27M mutation, said method comprising administering to the subject a composition of claim 60, in an amount effective to treat the DMG in the subject.


SEQUENCE LISTING (42 sequences)

SEQ ID NO: 1 - protein, 135 aa, synthetic construct
  REGION 1..135: note = Synthetic Polypeptide
ARTKQTARKSTGGKAPRKQLATKAARMSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 2 - DNA, 3000 bp, synthetic construct
  misc_feature 1..3000: note = Synthetic Polynucleotide
  source 1..3000: mol_type = other DNA
ctaatacgactcactatagggagacaagcttatggctcgtacaaagcagactgcccgcaaatcgaccggtggtaaagcacccaggaagcaactggctacaaaagccgctcgcaagagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 3 - RNA, 502 nt, synthetic construct
  source 1..502: mol_type = other RNA
gggagacaagcttatggctcgtacaaagcagactgcccgcaaatcgaccggtggtaaagcacccaggaagcaactggctacaaaagccgctcgcaagagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaattcttaattaaaaaaaaaaaaaaaaaaaaaaaaaaaaaaaaaaaaaaaaaaaaaaaaaaaaaaaaaaaaaactag

SEQ ID NO: 4 - protein, 136 aa, synthetic construct
  REGION 1..136: note = Synthetic Polypeptide
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 5 - DNA, 3000 bp, synthetic construct
  misc_feature 1..3000: note = Synthetic Polynucleotide
  source 1..3000: mol_type = other DNA
ctaatacgactcactatagggagacaagcttatggctcgtacaaagcagactgcccgcaaatcgaccggtggtaaagcacccaggaagcaactggctacaaaagccgctcgcatgagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 6 - RNA, 502 nt, synthetic construct
  source 1..502: mol_type = other RNA
gggagacaagcttatggctcgtacaaagcagactgcccgcaaatcgaccggtggtaaagcacccaggaagcaactggctacaaaagccgctcgcatgagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaattcttaattaaaaaaaaaaaaaaaaaaaaaaaaaaaaaaaaaaaaaaaaaaaaaaaaaaaaaaaaaaaaaactag

SEQ ID NO: 7 - protein, 136 aa, synthetic construct
  REGION 1..136: note = Synthetic Polypeptide
MARTKQTARKSTGGKAPRKQLATKAARMSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 8 - DNA, 2670 bp, synthetic construct
  misc_feature 1..2670: note = Synthetic Polynucleotide
  source 1..2670: mol_type = other DNA
ctaatacgactcactatagggagacaagcttatggcacccaggaagcaactggctacaaaagccgctcgcaagagtgcgccctctactggaggggtgaagaaacctcattaagaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 9 - DNA, 172 bp, synthetic construct
  source 1..172: mol_type = other DNA
gggagacaagcttatggcacccaggaagcaactggctacaaaagccgctcgcaagagtgcgccctctactggaggggtgaagaaacctcattaagaattcttaattaaaaaaaaaaaaaaaaaaaaaaaaaaaaaaaaaaaaaaaaaaaaaaaaaaaaaaaaaaaaaactag

SEQ ID NO: 10 - protein, 26 aa, synthetic construct
  REGION 1..26: note = Synthetic Polypeptide
MAPRKQLATKAARKSAPSTGGVKKPH

SEQ ID NO: 11 - DNA, 2670 bp, synthetic construct
  misc_feature 1..2670: note = Synthetic Polynucleotide
  source 1..2670: mol_type = other DNA
ctaatacgactcactatagggagacaagcttatggcacccaggaagcaactggctacaaaagccgctcgcatgagtgcgccctctactggaggggtgaagaaacctcattaagaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 12 - RNA, 171 nt, synthetic construct
  source 1..171: mol_type = other RNA
ggagacaagcttatggcacccaggaagcaactggctacaaaagccgctcgcatgagtgcgccctctactggaggggtgaagaaacctcattaagaattcttaattaaaaaaaaaaaaaaaaaaaaaaaaaaaaaaaaaaaaaaaaaaaaaaaaaaaaaaaaaaaaaactag

SEQ ID NO: 13 - protein, 26 aa, synthetic construct
  REGION 1..26: note = Synthetic Polypeptide
MAPRKQLATKAARMSAPSTGGVKKPH

SEQ ID NO: 14 - RNA, 81 nt, synthetic construct
  source 1..81: mol_type = other RNA
atggcacccaggaagcaactggctacaaaagccgctcgcatgagtgcgccctctactggaggggtgaagaaacctcattaa

SEQ ID NO: 15 - protein, 21 aa, synthetic construct
  REGION 1..21: note = Synthetic Polypeptide
RKQLATKAARMSAPSTGGVKK

SEQ ID NO: 16 - protein, 5 aa, synthetic construct
  REGION 1..5: note = Synthetic Polypeptide
MSAPS

SEQ ID NO: 17 - protein, 5 aa, synthetic construct
  REGION 1..5: note = Synthetic Polypeptide
RMSAP

SEQ ID NO: 18 - protein, 5 aa, synthetic construct
  REGION 1..5: note = Synthetic Polypeptide
ARMSA

SEQ ID NO: 19 - protein, 5 aa, synthetic construct
  REGION 1..5: note = Synthetic Polypeptide
AARMS

SEQ ID NO: 20 - protein, 5 aa, synthetic construct
  REGION 1..5: note = Synthetic Polypeptide
KAARM

SEQ ID NO: 21 - RNA, 63 nt, synthetic construct
  source 1..63: mol_type = other RNA
aggaagcaactggctacaaaagccgctcgcatgagtgcgccctctactggaggggtgaagaaa

SEQ ID NO: 22 - protein, 22 aa, synthetic construct
  REGION 1..22: note = Synthetic Polypeptide
RKQLATKAARMSAPSTGGVKKP

SEQ ID NO: 23 - protein, 29 aa, synthetic construct
  REGION 1..29: note = Synthetic Polypeptide
GKAPRKQLATKAARMSAPSTGGVKKPHRY

SEQ ID NO: 24 - protein, 15 aa, synthetic construct
  REGION 1..15: note = Synthetic Polypeptide
GKAPRKQLATKAARM

SEQ ID NO: 25 - protein, 15 aa, synthetic construct
  REGION 1..15: note = Synthetic Polypeptide
KAPRKQLATKAARMS

SEQ ID NO: 26 - protein, 15 aa, synthetic construct
  REGION 1..15: note = Synthetic Polypeptide
APRKQLATKAARMSA

SEQ ID NO: 27 - protein, 15 aa, synthetic construct
  REGION 1..15: note = Synthetic Polypeptide
PRKQLATKAARMSAP

SEQ ID NO: 28 - protein, 15 aa, synthetic construct
  REGION 1..15: note = Synthetic Polypeptide
RKQLATKAARMSAPS

SEQ ID NO: 29 - protein, 15 aa, synthetic construct
  REGION 1..15: note = Synthetic Polypeptide
KQLATKAARMSAPST

SEQ ID NO: 30 - protein, 15 aa, synthetic construct
  REGION 1..15: note = Synthetic Polypeptide
QLATKAARMSAPSTG

SEQ ID NO: 31 - protein, 15 aa, synthetic construct
  REGION 1..15: note = Synthetic Polypeptide
LATKAARMSAPSTGG

SEQ ID NO: 32 - protein, 15 aa, synthetic construct
  REGION 1..15: note = Synthetic Polypeptide
ATKAARMSAPSTGGV

SEQ ID NO: 33 - protein, 15 aa, synthetic construct
  REGION 1..15: note = Synthetic Polypeptide
TKAARMSAPSTGGVK

SEQ ID NO: 34 - protein, 15 aa, synthetic construct
  REGION 1..15: note = Synthetic Polypeptide
KAARMSAPSTGGVKK

SEQ ID NO: 35 - protein, 15 aa, synthetic construct
  REGION 1..15: note = Synthetic Polypeptide
AARMSAPSTGGVKKP

SEQ ID NO: 36 - protein, 15 aa, synthetic construct
  REGION 1..15: note = Synthetic Polypeptide
ARMSAPSTGGVKKPH

SEQ ID NO: 37 - protein, 15 aa, synthetic construct
  REGION 1..15: note = Synthetic Polypeptide
RMSAPSTGGVKKPHR

SEQ ID NO: 38 - protein, 15 aa, synthetic construct
  REGION 1..15: note = Synthetic Polypeptide
MSAPSTGGVKKPHRY

SEQ ID NO: 39 - protein, 9 aa, synthetic construct
  REGION 1..9: note = Synthetic Polypeptide
RKQLATKAA

SEQ ID NO: 40 - protein, 9 aa, synthetic construct
  REGION 1..9: note = Synthetic Polypeptide
LATKAARMS

SEQ ID NO: 41 - protein, 9 aa, synthetic construct
  REGION 1..9: note = Synthetic Polypeptide
ARMSAPSTG

SEQ ID NO: 42 - protein, 9 aa, synthetic construct
  REGION 1..9: note = Synthetic Polypeptide
SAPSTGGVK